(19) United States
(12) Patent Application Publication
(10) Pub. No.: US 2004/0229211 A1
(43) Pub. Date: Nov. 18, 2004

(54) Sensitive diagnostic testing methodology using multiplex real time PCR with one dye (MOD) and its use as in severe acute respiratory syndrome (SARS)

(76) Inventor: Wah Hin Alex Yeung, Central (HK)

Correspondence Address: Yeung, Wah Hin Alex, 5/F, Club Lusitano, 16 Ice House Street, Central, HK

(21) Appl. No.: 10/455,043

(22) Filed: Aug. 22, 2003

Related U.S. Application Data
(60) Provisional application No. 60/469,812, filed on May 13, 2003.

(51) Int. Cl. C12Q 1/70; C12Q 1/68; C12P 19/34

(52) U.S. Cl. 435/5; 435/6; 435/91.2

(57) ABSTRACT

This invention provides a new and more sensitive methodology for multiplex real time PCR testing of any genetic materials. The method is best illustrated here with the detection and diagnosis of Severe Acute Respiratory Syndrome (SARS) but can be applicable to any other usage where more sensitivity is required. It can be combined with the usual multiplex real time PCR test for further confirmation and multiple parameter assessment of SARS or other diseases. A kit form is provided for the detection of SARS by this methodology, but equally applicable to other diseases or testing conditions.

### BACKGROUND ART

Multiplex real time quantitative PCR and RT-PCR have been used for the detection of a number of markers separately in biological specimens. The sensitivity of this method has never been an issue in the past as the stress has always been on the specificity. However, with the occurrence of the SARS epidemic in certain parts of the world, it has been apparent that sensitivity is indeed an issue to detect very minute or fragmented genetic materials in this disease. To be able to detect at a very low level a disease that is still in its incubation phase is vital for the control of its spread. We have seen the horrific outcome of SARS if a carrier is not identified in time. As RNA is labile and easily fragmented, there was considerable difficulty in the past to use the test for human diseases as compared with DNA methods. Tumor derived RNA was detected in cancer patients. Similar results were found by the detection of mRNA of the tyrosinase gene¹, telomerase components² and viral RNA³. Recently our laboratory was able to repeat the experiment showing that GAPDH RNA and other RNAs were stabilized by the association with particles in the plasma⁴. In this invention, a new multiplex real time PCR is utilized to increase the sensitivity of detecting the minutest amount of a biological specimen. We would like to use the SARS virus detection as an example of how this methodology works.

### BRIEF SUMMARY OF THE INVENTION

The present invention provides a new method for the detection and diagnosis of a biological specimen such as infection due to the SARS virus and other diseases, as well as monitoring and predicting the outcome of such diseases. Only in the recent past, viral infections were essentially diagnosed by the positive cultures of biological specimens collected from animals or patients. The process was cumbersome as well as time consuming. With the recent SARS virus infection that spread quickly far and wide globally in a matter of weeks, viral culture as a diagnostic test for SARS is clearly not an adequate measure. Real time quantitative RT-PCR was used⁵ to detect the presence of the SARS virus using primers derived from the coding region of the polymerase gene of the virus. This method probably lacks the sensitivity required to detect an early case or in which the viral load is small (inventor's experience with researchers testing SARS clinical samples). This invention is to overcome this difficulty. The biological specimen may come from the nasal or throat swab of a SARS suspected patient. Alternatively, blood or stool samples can also be used. The buffy coat of blood, containing most of the white cells, will also be an important source of testing genetic materials since the inventor believes that the SARS virus may be hidden in the lymphocyte fraction of the white cells in early asymptomatic cases. The sensitivity of this invention, tested in our laboratory, is at least many folds more than the usual one using the polymerase transcript. If this fact is further proven by clinical practice, this methodology will then have a good chance to detect early SARS carriers. The usefulness of detecting early or asymptomatic carriers is obvious in the control of this devastating illness.

### DESCRIPTION OF FIGURES AND DIAGRAM

FIG. 1: Transcriptional organisation of a typical coronavirus. All transcripts share coincident polyadenylated 3′ ends, and splice variation is achieved via nested transcriptional start sites (ref. 2). A RT-PCR test targeting the 3′ end will thus target all potential transcripts whereas one targeting the 5′ end will not.

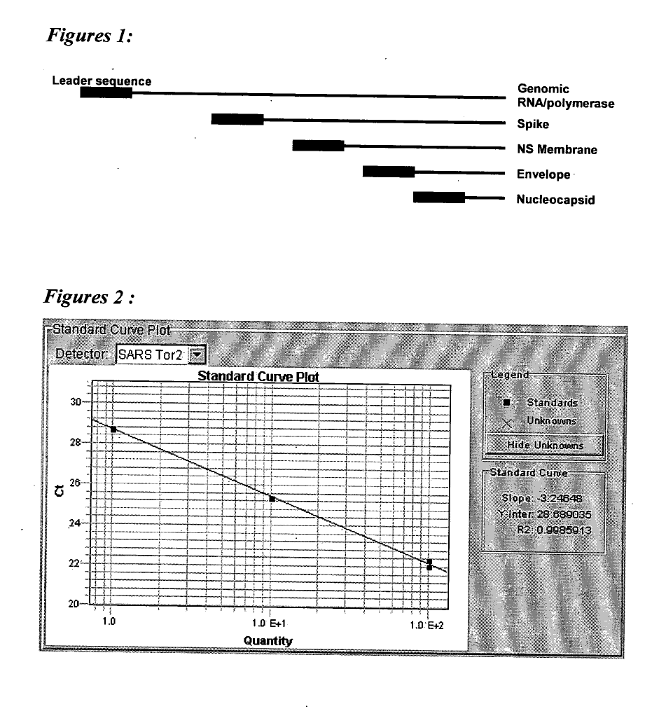

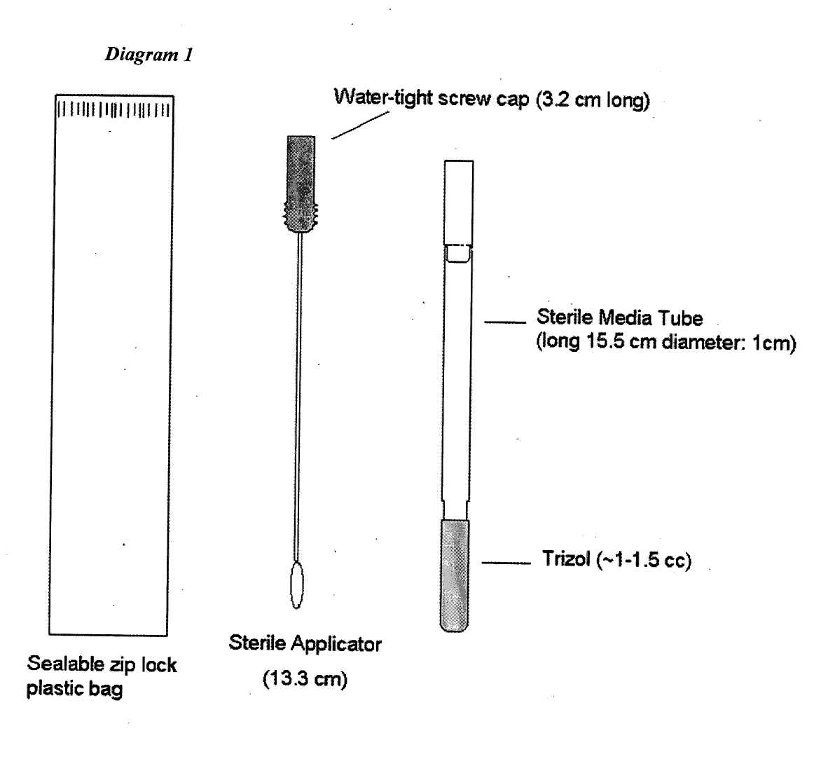

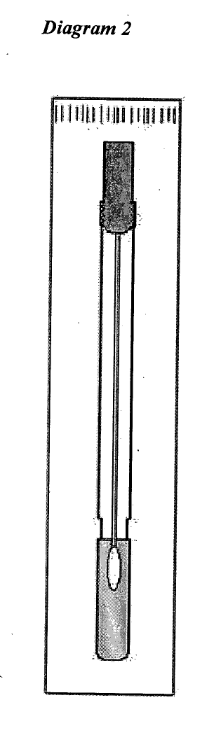

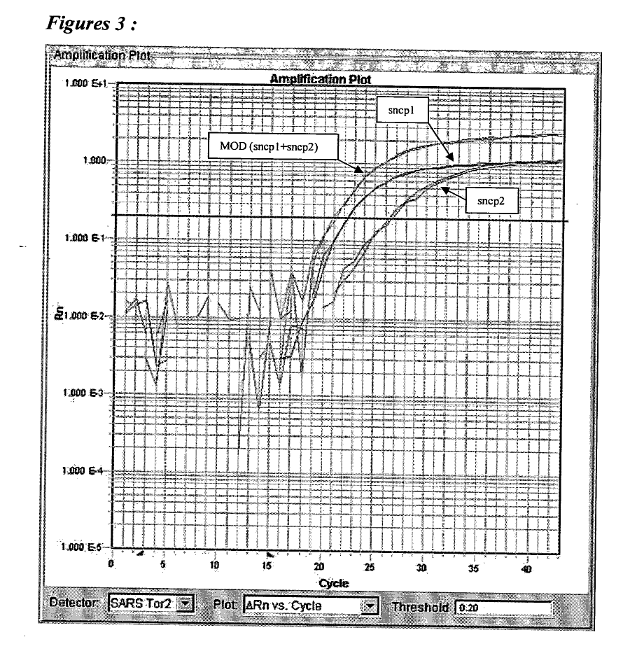

Diagram 1: a test tube for use with a throat swab and with trizol of 1 to 1.5 cc at the bottom of the tube as a kit before use

Diagram 2: The kit after use being put into a zip-lock bag for transportation to the laboratory for analysis

### DETAILED DISCLOSURE OF THE INVENTION

Theory of the Invention

The general principle of PCR is well known to the art and is widely used. However the basic methodology has several disadvantages that are mostly linked with the detection of the amplification products by gel electrophoresis. It requires additional handling of the sample which is time-consuming and labor intensive. It is also more prone to mix-ups. The sensitivity is low and quantification is difficult, if not impossible. Real time PCR uses the method in which the DNA generated within a PCR is detected on a cycle-by-cycle basis during the PCR reaction. The amount of DNA increases the faster the more template sequences are present in the original sample. When enough amplification and the DNA so amplified exceeds a threshold the PCR product is identified. Most instruments that are used for a real time PCR detect an increase of fluorescence of a specific wave length as a result of an increasing amount of the PCR product. For example, the Applied BioSystems Prism 7700 sequence detection system is based on the combination of PCR and hybridization of a fluorogenic, target specific probe. The probe itself is an oligonucleotide with both a reporter and a quencher dye attached at the 5′ and 3′ end respectively. The fluorescence of the reporter dye is efficiently quenched by the quencher dye as long as both fluorochromes are present in close proximity. If the target sequence is present, the probe anneals between the forward and reverse primers. During PCR amplification and thus elongation of the primers the probe is cleaved by the 5′ nuclease activity of the DNA polymerase. This process separates the reporter dye from the quencher dye, making the reporter detectable. Since these reporter molecules proportionally increase with each cycle, an algorithm of the software of the instrument comparing the simultaneous increase of reporter and decrease of the quencher dye once every few seconds during the PCR reaction will generate a normalized signal. The first cycle in which the normalized reporter signal is above a defined threshold is named as the threshold cycle. This threshold cycle value is proportional to the copy number of the template used for quantification. If the template is not DNA but RNA, a real time reverse transcription (RT) PCR is performed. In this case, the RNA is first transcribed into cDNA before the actual real time PCR is performed.

If two or more target sequences are amplified simultaneously in the same PCR reaction, a multiplex PCR is performed. In this invention, since this is targeted towards sensitivity of detection, the two or more sets of primers are targeting different regions of the same or different genes. As contrary to the conventional multiplex real time PCR, the probe sets use the same reporter and quencher dye. In theory, if the biological specimen contains one gene that have two or more regions targeted by different sets of primers, the multiplex real time PCR with one dye (MOD) in this invention will detect a total sum of all of the increase of the primer sets and will not be able to differentiate which quantity with which specific sequence it is amplifying. If the number of primer sets is (n), then the quantity of the MOD system will report n x the true starting copy number of the template gene. However, because of the proportional increase of the reporter signal, the threshold cycle will be lowered for many folds and hence, an increase of sensitivity. In another scenario where only fragments of a gene are expected to be present, the simultaneous multiplexing with different primers targeting the different regions of the gene will be able to have a much higher chance of gene detection. In SARS infection, if only sub-genomic fractions are present in the early going, then targeting one region with the conventional PCR may be an insensitive tool. In yet another scenario, RNA fragments are labile and destroyed by the bodily fluids in which they are transported. Partial destruction will lead to even more sub-genomic fractions suitable for detection by this invention. In summary, whether the biological specimen contains the minutest genetic material for testing, or whether the starting material is fragmented to start with, or fragmented due to enzyme digestion or other routes of destruction, or whether the starting material may be constantly shifting its genetic transcription due to mutation or from different strains, the MOD technique is the sensible as well as sensitive answer for its detection.

Apart from a very sensitive methodology for detection of genetic materials, MOD may also be useful, combining with the usual multiplex real time PCR using different probe reporter dyes, as a confirmation test of certain difficult to diagnose diseases. SARS infection is one of these. In SARS infection, mutation and constant changes of genetic transcription may be taking place. The two step process of MOD and the usual multiplex real time PCR may be able to detect such a mutation and transformation. For example, one might detect the virus by MOD and then upon further normal multiplexing, the usual S glycoprotein is not present. A mutation or transformation may be present in this case and further gene sequencing may be necessary to document the process. The two step process may also be able to stage the infective process. For example, certain stage of the infection may be revealed by a differential composition and quantity of the different proteins of the virus. It may also be able to accurately measure the viral load leading to establishing the prognosis of the patient. This serves as a guide in treatment choice and opportunities.

Material and Method

Illustrated with an Example of the SARS Virus RNA

Cellular culture supernatant of SARS virus-infected Vero E6 cells was collected at the Chinese University of Hong Kong and RNA was extracted using a QIAamp Viral RNA Mini Kit (Qiagen, Hilden, Germany). We are comparing the relative sensitivity of detecting viral RNA from cell culture supernatant and RNA stemming from a clinical specimen from a SARS patient, extracted using various standard techniques depending on the specimen in question.

Processing of Blood Samples

We analyse either the plasma or the buffy coat derived RNA extracted from the blood of SARS-patients. 5 ml of blood is collected in EDTA tubes from patients satisfying the clinical definition of SARS. After centrifugation at 1600 g (Beckman, GPR centrifuge, USA) for 10 minutes, the buffy coat layer is removed with a transfer pipette. The isolated cells are washed with PBS buffer (Invitrogen Corporation, N.Y. USA), spun down and the buffy coat layer is removed and transferred to a fresh micro-centrifuge tube where it is re-suspended in 200 μl of PBS buffer (Invitrogen). The process is similar for plasma except that the portion extracted is the plasma fraction of the blood and not the buffy coat. RNA is extracted using an RNeasy Mini Kit (Qiagen) following the manufacturers instructions.

Real-Time Quantitative RT-PCR

A one-step real-time quantitative RT-PCR method was used for RNA quantification of the SARS virus on an ABI Prism 7900HT Sequence Detection System (Applied Biosystems). We first used the different regions of the nucleocapsid protein. The viral polymerase region was also used as an alternative RT-PCR target as a test of relative RT-PCR sensitivity when targeting different viral regions. The primers for all of the RT-PCR assays were designed from the TOR2 strain of the SARS coronavirus (National Centre for Biotechnology Information website: www.ncbi.nlm.nih.gov/), using Primer Express software version 2.0 (Applied Biosystems, Foster City, Calif.). Primer sets were specifically chosen so as to encompass the Replicase Polyprotein region (Pol 1) and the whole nucleocapsid coding region. The choice of using the nucleocapsid coding region is to maximize the possibility of a sub-genomic or sub-transcriptional fragment being amplified. The primer and fluorescent probe sequences used for amplification are summarized here for Pol 1 and in Table 1 for the nucleocapsid protein.

Name:

| | |
|---|---|
| Pol 1 | |
| 1. SARS1 forward primer: | |
| TTA TCA CCC GCG AAG AAG CT | (SEQ ID NO: 1) |
| 2. SARS2 reverse primer: | |
| CTC TAG TTG CAT GAC AGC CCT C | (SEQ ID NO: 2) |
| 3. SARP probe: | |
| TET-TCGTGCGTGGATTGGCTTTGATGT-TAMRA | (SEQ ID NO: 3) |

1. SARS1 18187-18206 Tm: 58.3 degree C.

2. SARS2 18264-18243 Tm: 57.5 degree C.

3. SARSP 18218-18241 Tm: 68.1 degree C.

For the MOD methodology, the Pol 1 SARP probe uses TET as the reporter dye and in the normal multiplex real time PCR methodology, the Pol 1 SARP probe uses any other dye other than the FAM, TET, VIC, or HEX used by the four nucleocapsid protein primer sets described in Table 2.

Table 1. Primers and probes designed to target the nucleocapsid region of the TOR2 isolate of SARS coronavirus. Primer sets were specifically designed to encompass the whole nucleocapsid region. Each probe is conjugated to the same reporter dye, TET, in this case. This strategy was devised in order to increase the total signal generated by sub-genomic or low abundance PCR transcripts. Any reporter dye could theoretically be adopted.

Table 2. Primers and probes designed to target the nucleocapsid region of the Tor2 isolate of SARS coronavirus. Primer sets were specifically designed to encompass the whole nucleocapsid region. Each probe is conjugated to a different reporter dye.

The Initial Set Up

Calibration curve quantification was prepared by serial dilution of SARS virus infected cell culture supernatant RNA (as above). Concentrations ranged from 60 ng to 20 pg. Concentrations were expressed as weight of extracted RNA per RT-PCR well.

The RT-PCR reactions were set up according to the manufacturer's instructions except that reactions were scaled down to use reaction volumes of 25 μl with all components, except the fluorescent probes and amplification primers, being obtained from an EZ rTth RNA PCR reagent set (Applied Biosystems, Foster City, Calif.). The fluorescent probes were custom-synthesized by Proligo (Singapore) and were used at concentrations of 100 nM. The PCR primers were synthesized by Proligo and were used at a concentration of 200 nM. Volumes of 1 μl of the diluted, extracted supernatant RNA were used for amplification. Each sample was analyzed in duplicate, and the corresponding calibration curve was run in parallel for each analysis. Samples are also tested to ensure they are negative for DNA by substituting the rTth polymerase with the AmpliTaq Gold enzyme (Applied Biosystems, Foster City, Calif.) which is devoid of reverse transcriptase activity. In addition, multiple negative water blanks were included in every analysis.

The thermal profile used for the analysis was as follows: the reaction was initiated at 50° C. for 2 min in the presence of the enzyme uracil N-glycosylase, which destroys any contaminating uracil-containing PCR amplicons. This step was followed by a reverse transcription step at 60° C. for 30 min. After a 5-min denaturation at 95° C., PCR was carried out for 43 cycles using a denaturation step of 94° C. for 20 s and a combined annealing/extension step set at the temperature appropriate for the appropriate temperature of the primer sets. The following is an example of the one done for the nucleocapsid proteins.

FIG. 2. Calibration curve of various 10-fold dilutions of SARS viral DNA quantified by real-time RT-PCR on an ABI 7900 sequence detection system targeting the viral nucleocapsid region. The amount of input RNA ranges between 60 ng and 20 pg per reaction.

The Real Time Multiplexing in One Dye Method (MOD)

For our multiplex RT-PCR thermal profile as described in our invention as the MOD technology, conditions are identical except for the addition of more primer sets (200 nM each) with the appropriate probes (100 nM) conjugated to the same reporter dye (the combined multiplex of sncp1 with sncp2 versus sncp1 alone and sncp2 alone as illustrated in FIG. 3) or it can be of any number combinations of primers as listed (Pol 1 and the nucleocapsid proteins in table 1). SDS software 2.0 (Applied Biosystems) interprets the total wavelength generated by the ABI prism 7900HT and calculates the total abundance of all the different RT-PCR amplicons. The result indicated that the MOD technology with sncp1 plus sncp2 was about two times more sensitive than using either sncp1 or sncp2 alone.

FIG. 3 showing the combining of sncp1 and sncp2 multiplex has at least a two times or more quantity of sncp1 or sncp2 alone.

The Standard Multiplex Real Time PCR

For our multiplex RT-PCR thermal profile,⁶ conditions are identical except for the addition of more primer sets (200 nM each) and the corresponding fluorescent probes (100 nM) conjugated to the different dyes of Pol 1 and the various nucleocapsid proteins (table 2). SDS software version 2.0 (Applied Biosystems) interprets the different wavelength data generated by the ABI Prism 7900HT and calculates the relative abundance of each different RT-PCR amplicon accordingly.

### CONCLUSION

Multiplexing with one dye (MOD) technology gave a result that was approximately as many folds more sensitive than the normal real time PCR technology using only one primer set and probe as the number of different primers and probes put into the test.

Reasoning of the Methodology

Our reasons for the MOD technology are numerous. We are aiming to maximize the sensitivity of our assay to produce a sufficiently sensitive and robust test to be of genuine practical value in almost any fields that are applicable. We would like to use the detection and diagnosis of SARS as a prime example for this MOD technology.

We would like to detect SARS virus RNA in specimens from pre-symptomatic or very early stage patients. In these scenarios, viral RNA will be collected in clinical specimens in various conditions including but not limited to whole, extracellular viral genomes; partial, damaged viral genomes; intracellular, replicating viral material; RNA polymerase-generated mRNA; and ribonuclease-degraded fragments of viral genomes, -ve sense transcripts and mRNAs.

Accordingly, our design strategy is as follows:

1) Because all coronavirus transcripts terminate at the 3′ end⁷, all transcripts, irrespective of the encoded protein, contain the 3′ nucleocapsid-coding region targeted by our RT-PCR primers. The sensitivity of this protocol in detecting replicating virus should thus be superior to protocols targeting the polymerase (Pol)-coding region only even without the new invention. Our MOD technology for SARS is likely to contain both the Pol region as well as the nucleocapsid region. Essentially the sensitivity will be increased many folds by adding the number of nucleocapsid transcripts and Pol, whereas the old method will be just from the transcripts showing Pol alone. In addition, the other regions such as the spike, envelope and membrane may also be incorporated easily to those skilled in the art, adding to even better sensitivity.

2) Our primer and probe sets were specifically chosen to produce PCR products of short length (67-80 bp). This design strategy will minimize the loss of target sequence due to host cell endonuclease activity and other viral genetic damage, and will ensure detection of the maximum number of subgenomic fragments present in a clinical specimen. However, longer length PCR products can obviously be incorporated into the MOD technology if so desired.

3) Transcription of the nucleocapsid region and glycoprotein like the envelope is likely to be a late-stage event in the viral replication cycle at a period when infected cells are likely to be releasing their contents (including viral mRNAs) into the surroundings. This viral RNA release may be due to virally damaged lytic cells or host cytotoxic T-cell-mediated cell death, either of which will result in clinical specimens relatively enriched with these envelope or nucleocapsid mRNAs. Sub-genomic fractions and damaged fractions may be abundant in addition to the number of increased transcripts as mentioned in 1). The best way to capture the detection of these is with multiplexing, and multiplexing with one dye for increased sensitivity.

4) As mentioned previously, MOD technology combining with the standard multiplex real time PCR will be able to detection mutation, different virus strains and staging of the infection. Confirmation of SARS infection is also instantly achieved by the second part of the standard multiplex test showing most, if not all of the different regions to be positive. A transcient bystander infection or contaminated samples with other strains of conora virus may show a positive Pol region test but not positivity in other regions. This confirmation is most important in the management of SARS.

Fields of Use

The MOD technology, with or without the accompanying standard multiplex real time PCR to further delineate and quantify the individual components of the biological specimen, has extended fields of usage.

The MOD technique and invention here can be applied to many human diseases where detection can be difficult at an early stage and especially when treatment is available at that stage. Cancer genes or cancer associated genes in pre-malignant diseases or early malignant diseases can be detected for an effective cure. Similarly these genetic alterations, if specific enough, may be detected at such an early stage of recurrence that effective treatment can be instilled again. A good example is the EBV DNA testing of nasopharyngeal cancer whereby a sensitive test may mean that the local or even distant metastasis at the early recurrence stage can be effectively cured. Certain genetic markers in leukemia and different types of lymphomas may also be targets for this invention. For other infections, detection of SARS and HIV virus during incubation period is vital for the control of the disease as well as treatment.

The MOD technique can also be used in the testing and sampling of live stock for diseases. For example, a sampling set for the SARS virus or H5N1 flu virus can be done during exporting or importing of live stocks, fresh or frozen food. The MOD technique is effective even mutation has set in since it does cast a wide and effective net to capture any part that has not undergone mutation.

The extreme sensitivity of the MOD system can be utilized in bio-terrorism, when extremely small sample quantity coupled with sometimes only fragments of the original molecule need to be detected in a fast and efficient manner. We can envision that using primers and probes specific for many different biological agents and their respective fragments be used as an initial screening of any material that is suspicious. If positive, then the standard multiplex test can then be used set by set for finding out the real culprit.

The MOD system can also be used in criminology for the same reasons above.

The MOD system can also be envisioned to be helpful in high throughput screening or screening of materials of unknown origin. Panels of different sets of primers and probes can be squeezed into chip sets or multiple chambers, making analysis of hundred or thousands of genetic materials an easier task than before.

The above described will not be a complete list of the different fields suitable for this invention. It will be easy for those skilled in the art to apply a sensitive detection test such as this into multiple and useful applications that could improve our daily life.

### REFERENCES

1. Kopreski M S, Benko F A, Kwak L W, Gocke C D. Detection of tumor messenger RNA in the serum of patients with malignant melanoma. Clin Cancer Res 1999;5:1961-5.

2. Dasi F, Liedo S, Garcia-Granero E, Ripoll R, Marugan M, Tormo M et al. Real-time quantification in plasma of human telomerase reverse transcriptase (hTERT) mRNA: a simple blood test to monitor disease in cancer patients. Lab Invest 2001;81:767-9

3. Lo K W, Lo Y M D, Leung S F, Tsang Y S, Chan L Y, Johnson P J, et al. Analysis of cell free Epstein-Barr virus associated RNA in the plasma of patients with nasopharyngeal carcinoma. Clin Chem 1999;45:1292-4.

4. Enders K O, Nancy B YT, Lam Y L, Lo Y M D et al. Presence of Filterable and non filterable mRNA in the plasma of cancer patients and healthy individuals. Clin Chem 2002;48:1212-17.

5. Peiris J S M, Lai S T, Poon L L M, Yuen K Y et al Coronavirus as a possible cause of severe acute respiratory syndrome. Lancet 2003; online publication Apr. 8 2003.

6. Wittwer C T, Herrmann M G, Gundry C N, Elenitoba-Johnson K S. Real-time multiplex PCR assays. Methods 2001 December;25(4):430-42

7. Sawicki S G, Sawicki D L. A new model for coronavirus transcription. Adv Exp Med Biol 1998;440:215-9 Related Articles, Links

TABLE 1

| | | Sense | Antisense | | Amplicon |
|---|---|---|---|---|---|
| Name | 5′ Start | primer (5′-3′) | primer (5′-3′) | Fluorescent Probe (5′-3′) | length |
| sncp1 | 28118 | GACCCCAATCAA | TCCATTCTGGTTATTGT | (TET)-CCCCCCGCATTACATTT | 80 |
| | | ACCAACGT | CAGTTGAA | GGTGGA-(TAMRA) | |
| | | (SEQ ID NO: 4) | (SEQ ID NO: 5) | (SEQ ID NO: 6) | |
| sncp2 | 28516 | GGAGCCTTGAAT | GCACGGTGGCAGCATTG | (TET)-CCACATTGGCACCCGCA | 67 |
| | | ACACCCAAAG | (SEQ ID NO: 8) | ATCCTAAT-(TAMRA) | |
| | | (SEQ ID NO: 7) | | (SEQ ID NO: 9) | |
| sncp3 | 29001 | CAAACATTGGCC | CAATGCGTGACATTCCA | (TET)-CACAATTTGCTCCAAGT | 68 |
| | | GCAAATT | AAGA | GCCTCTGC-(TAMRA) | |
| | | (SEQ ID NO: 10) | (SEQ ID NO: 11) | (SEQ ID NO: 12) | |
| sncp4 | 29265 | AAAGAAGCAGCC | GGAATTTTGAAGTTGTC | (TET)-TTCTTCCTGCGGCTGAC | 73 |
| | | CACTGTGACT | TGGAGAAA | ATGGATG-(TAMRA) | |
| | | (SEQ ID NO: 13) | (SEQ ID NO: 14) | (SEQ ID NO: 15) | |

TABLE 2

| | | Sense | Antisense | | Amplicon |
|---|---|---|---|---|---|
| Name | 5′ Start | primer (5′-3′) | primer (5′-3′) | Fluorescent Probe (5′-3′) | length |
| sncp1 | 28118 | GACCCCAATCAA | TCCATTCTGGTTATTGT | (FAM)-CCCCCCGCATTACATTT | 80 |
| | | ACCAACGT | CAGTTGAA | GGTGGA-(TAMRA) | |
| | | (SEQ ID NO: 4) | (SEQ ID NO: 5) | (SEQ ID NO: 16) | |
| sncp2 | 28516 | GGAGCCTTGAAT | GCACGGTGGCAGCATTG | (TET)-CCACATTGGCACCCGCA | 67 |
| | | ACACCCAAAG | (SEQ ID NO: 8) | ATCCTAAT-(TAMRA) | |
| | | (SEQ ID NO: 7) | | (SEQ ID NO: 17) | |
| sncp3 | 29001 | CAAACATTGGCC | CAATGCGTGACATTCCA | (VIC)-CACAATTTGCTCCAAGT | 68 |
| | | GCAAATT | AAGA | GCCTCTGC-(TAMRA) | |
| | | (SEQ ID NO: 10) | (SEQ ID NO: 11) | (SEQ ID NO: 18) | |
| sncp4 | 29265 | AAAGAAGCAGCC | GGAATTTTGAAGTTGTC | (HEX)-TTCTTCCTGCGGCTGAC | 73 |
| | | CACTGTGACT | TGGAGAAA | ATGGATG-(TAMRA) | |
| | | (SEQ ID NO: 13) | (SEQ ID NO: 14) | (SEQ ID NO: 19) | |

What is claimed is:

1. A method for detecting and diagnosing any genetic material sample using a multiplex real time PCR technique with two or more primers but with their corresponding specific probe sets using the same reporter dye. This multiplex with the same one dye technique is referred as “MOD” technology. The primer sets are designed to target:
(a) the same or different regions of the same gene in the biological sample. The method can, if necessary, be followed
(b) by a confirmatory multiplex real time PCR with different probe dyes to determine the true composition and quantities of the different markers.

2. The method of claim 1, wherein the regions of the gene can be of two or more different genes in the biological sample.

3. The method of claim 1, wherein the gene or genetic materials can be of any origin, DNA, RNA (using RT-PCR) or otherwise, so long it fits the criteria for amplification purposes by those skilled in the art.

4. The method of claim 1, wherein the gene can be of any size or fragment.

5. The method of claim 1, wherein the gene is a cellular gene.

6. The method of claim 1, wherein the gene is an endogenous gene.

7. The method of claim 1, wherein the gene is a transgene.

8. The method of claim 1, wherein the gene is a viral gene.

9. The method of claim 1, wherein the methodology is not limited to real time PCR such as sybr green, TaqMan real time PCR or RT-PCR, but can be of any methodology used by those skilled in the art wherein the collective use of multiple markers with the same reporting agent is for the purpose of increasing sensitivity of the test.

10. The method of claim 1, wherein the methodology is used for the diagnosis of pre-malignant conditions or cancer.

11. The method of claim 1, wherein the methodology is used in the criminology field for ultra sensitive detection of minute or fragmented genetic materials.

12. The method of claim 1, wherein the methodology is used in the detection of bio-terrorism materials with minute or fragmented samples.

13. The method of claim 1, wherein the field of use is in animals instead of human.

14. The method of claim 1, wherein the field of use is in plants and agriculture.

15. A method for detecting and diagnosing any genetic material sample using a multiplex real time PCR technique with two or more primers but with their corresponding specific probe sets using the same reporter dye. This multiplex with the same one dye technique is referred as “MOD” technology. The primer sets are designed to target the same or different regions of the same gene in the biological sample. The method can, if necessary, be followed by a confirmatory multiplex real time PCR with different probe dyes to determine the true composition and quantities of the different markers, wherein the methodology is used in the detection and diagnosis of Severe Acute Respiratory Syndrome virus (SARS) infection carriers or patients.

16. The method of claim 15, wherein the methodology is used for the confirmation, mutation status, staging, viral strain, viral quantity and prognosis of SARS patients.

17. The method of claim 15 in a diagnostic kit, consisting of different primer sets for different regions of the same gene or different genes and the corresponding probes all in one reporter dye, regardless of mechanism and types.

18. The method of claim 15, the diagnostic kit is with the same primer sets but with the corresponding probe sets with different reporter dyes, regardless of mechanism and types.

19. The method of claims 15, wherein different primer sets for different regions of the same gene or different genes and the corresponding probes all in one dye and also in different dyes.

20. The method of claims 15, wherein the different primer sets target one of the genes of the SARS vrius.

21. The method of claim 15, wherein the different primer sets target at least two to all of the genes of the SARS virus, namely the replicase polyprotein (Pol) region, the spike (S), membrane (M) and envelope (E) glycoprotein, and the nucleocapsid protein (N), or any yet discovered region.

22. The method of claims 15, wherein the biological samples are from all the usual sources known to those skilled in the art such as swab or lavage from nasal, nasopharynx and throat, blood plasma or serum, urine, stool, pleural or bronchial aspirate etc.

23. The method of claims 15, wherein the biological specimen comes only from the white blood cell or buffy coat of the blood sample.

24. The method of claims 15, wherein the biological specimen comes from the particle fractions of the blood where RNA is associated (referenced U.S. Application 60/380,708).

25. The method of claims 15, wherein the detection and diagnosis is for other infectious diseases besides SARS.

26. A method for detecting and diagnosing any genetic material sample using a multiplex real time PCR technique with two or more primers but with their corresponding specific probe sets using the same reporter dye. This multiplex with the same one dye technique is referred as “MOD” technology. The primer sets are designed to target the same or different regions of the same gene in the biological sample. The method can, if necessary, be followed by a confirmatory multiplex real time PCR with different probe dyes to determine the true composition and quantities of the different markers, wherein the methodology is used in the detection and diagnosis of Severe Acute Respiratory Syndrome virus (SARS) infection carriers or patients, wherein the kit contains separately or in combination with specially designed nasal or throat swab tubes as illustrated.


SEQUENCE LISTING (19 sequences)

SEQ ID NO: 1 - DNA, 20 bp, Artificial
  SARS1 forward primer
ttatcacccgcgaagaagct

SEQ ID NO: 2 - DNA, 22 bp, Artificial
  SARS2 reverse primer
ctctagttgcatgacagccctc

SEQ ID NO: 3 - DNA, 24 bp, Artificial
  SARP probe
ncgtgcgtggattggctttgatgn

SEQ ID NO: 4 - DNA, 20 bp, Artificial
  sncp1 forward primer
gaccccaatcaaaccaacgt

SEQ ID NO: 5 - DNA, 25 bp, Artificial
  sncp1 reverse primer
tccattctggttattgtcagttgaa

SEQ ID NO: 6 - DNA, 23 bp, Artificial
  sncp1 probe
ncccccgcattacatttggtggn

SEQ ID NO: 7 - DNA, 22 bp, Artificial
  sncp2 forward primer
ggagccttgaatacacccaaag

SEQ ID NO: 8 - DNA, 17 bp, Artificial
  sncp 2 reverse primer
gcacggtggcagcattg

SEQ ID NO: 9 - DNA, 25 bp, Artificial
  sncp2 probe
ncacattggcacccgcaatcctaan

SEQ ID NO: 10 - DNA, 19 bp, Artificial
  sncp3 forward primer
caaacattggccgcaaatt

SEQ ID NO: 11 - DNA, 21 bp, Artificial
  sncp3 reverse primer
caatgcgtgacattccaaaga

SEQ ID NO: 12 - DNA, 25 bp, Artificial
  sncp3  probe
nacaatttgctccaagtgcctctgn

SEQ ID NO: 13 - DNA, 22 bp, Artificial
  sncp4 forward primer
aaagaagcagcccactgtgact

SEQ ID NO: 14 - DNA, 25 bp, Artificial
  sncp4 reverse primer
ggaattttgaagttgtctggagaaa

SEQ ID NO: 15 - DNA, 24 bp, Artificial
  sncp4 probe
ntcttcctgcggctgacatggatn

SEQ ID NO: 16 - DNA, 23 bp, Artificial
  sncp1 probe
ncccccgcattacatttggtggn

SEQ ID NO: 17 - DNA, 25 bp, Artificial
  sncp2 probe
ncacattggcacccgcaatcctaan

SEQ ID NO: 18 - DNA, 25 bp, Artificial
  sncp3 probe
nacaatttgctccaagtgcctctgn

SEQ ID NO: 19 - DNA, 24 bp, Artificial
  sncp4 probe
ntcttcctgcggctgacatggatn